(19) United States
(12) Patent Application Publication
(10) Pub. No.: US 2022/0040067 A1
(43) Pub. Date: Feb. 10, 2022

(54) MATT-EFFECT COSMETIC COMPOSITION

(71) Applicant: L'OREAL, Paris (FR)

(72) Inventors: Sylvie JALENQUES, Chevilly Larue (FR); Elena CATALAN-MARTIN, Chevilly Larue (FR); Chloe LAIDBOEUR, Chevilly Larue (FR)

(73) Assignee: L'OREAL, Paris (FR)

(21) Appl. No.: 17/415,117

(22) PCT Filed: Dec. 20, 2019
(86) PCT No.: PCT/EP2019/086715

§ 371 Date: Jun. 17, 2021

(30) Foreign Application Priority Data
Dec. 21, 2018 (FR) 1873735

(51) Int. Cl. A61K 8/25; A61K 8/06; A61Q 1/12; A61K 8/37

(52) U.S. Cl. CPC A61K 8/25; A61K 8/062; A61K 2800/412; A61K 8/375; A61Q 1/12

(57) ABSTRACT

The invention relates to a composition, notably a cosmetic and/or dermatological composition, in particular for caring for a keratin material, notably the skin and in particular facial skin, which is in the form of an oil-in-water, O/W, emulsion, and comprising at least a) particles of at least one hydrophobic silica aerogel, b) an ester hydrocarbon-based oil, and c) an ether hydrocarbon-based oil, in which said ether and ester oils are present in an ether oil/ester oil weight ratio ranging from 1 to 2.

### TECHNICAL FIELD

The present invention relates to the field of caring for keratin materials, notably the skin, and relates more particularly to a composition, notably a cosmetic and/or dermatological composition, for caring for the skin and notably which is most particularly effective for making facial skin matt and/or reducing its shininess.

### PRIOR ART

Obtaining a matt effect on the skin is highly desired by users, notably users with combination or greasy skin. The reason for this is that these skin types are very often associated with greater secretion of sebum, which produces an undesirable shiny effect on the surface of the skin. Moreover, skin on which excessive secretion of sebum takes place may constitute a favourable medium for the proliferation of normally harmless bacteria which live on the skin and then give rise to skin imperfections such as spots or rashes. These unesthetic and thus undesirable manifestations are particularly exacerbated in the case of adolescents, but may also occur in adults, notably as a consequence of hyperproduction of androgens or as a consequence of external factors such as pollution. Finally, it is also known that shiny skin generally gives rise to poorer staying power of makeup, which thus has a tendency to become degraded over the course of the day.

Galenical formulations intended for skincare and more particularly for adjusting shininess have thus already been proposed. In general, they comprise a “sebum-regulating” agent, i.e. an agent that is capable of aiding in regulating the activity of the sebaceous glands by means of an action that may be termed biological, and/or one or more sebum-absorbing fillers, for instance perlite (FR 2881643) and fumed fillers (EP 1 637 186). However, these fillers may lead to discomfort, notably fluff on the skin and/or a perception of having unclean skin.

More recently, the Proprietor has noted that hydrophobic aerogels also prove to be effective for improving the mattness of the skin in a persistent manner. However, the use of this type of material, in a weight amount of greater than 1%, proves to be detrimental to the sensory qualities of compositions in which they are incorporated. On application to the skin, these compositions are judged as being excessively dragging, tacky and with a coarse skin finish. The perception of moisturization is poor, immediately and in the long term, and thus at variance with user expectations with regard to galenical formulations of this type.

There is thus still a need for cosmetic compositions which afford an effective (strong), immediate and long-lasting matt effect while at the same time remaining endowed with satisfactory cosmetic properties such as a glidant, non-tacky texture on application with a clean and non-coarse skin finish and which are capable of affording immediate and long-term moisturizing and clean skin sensations.

### DISCLOSURE OF THE INVENTION

The object of the present invention is, precisely, to satisfy this expectation.

Thus, the present invention is mainly directed towards a composition, notably a cosmetic and/or dermatological composition, for caring for a keratin material, notably the skin and in particular facial skin, which is in the form of an oil-in-water, 0/W, emulsion, and containing at least

a) particles of at least one hydrophobic silica aerogel,

b) an ether hydrocarbon-based oil, and

c) an ester hydrocarbon-based oil,

said ether and ester hydrocarbon-based oils being present in an ether oil/ester oil weight ratio ranging from 1 to 2.

As emerges from the examples below, a composition according to the invention makes it possible to improve the matt effect of the skin in a persistent manner. The skin thus durably maintains a matt effect. Besides this efficacy with regard to the shininess of the skin, the compositions according to the invention afford the sensory properties expected by users, on application and on wearing, i.e. a fluid, glidant, non-greasy and moisturizing feel on application, and this moisturizing feel and non-tacky effect remain over time.

According to a preferred embodiment, a composition according to the invention contains, as aerogel, at least one hydrophobic silica aerogel, in particular a silylated silica aerogel (INCI name: silica silylate).

According to another preferred embodiment, a composition according to the invention has, as solids, a content ranging from 0.5% to 13%, preferably a content ranging from 1% to 5% by weight, in particular ranging from 1% to 3% by weight of particles of at least one hydrophobic silica aerogel and in particular of a silylated silica aerogel (INCI name: silica silylate) relative to its total weight.

According to a preferred variant, a composition according to the invention contains less than 1% by weight, or even less than 0.5% by weight, and preferably is free of a silicone oil and/or a silicone elastomer notably of polysiloxane type.

According to another of its aspects, the present invention relates to a cosmetic process for the care, in particular the non-therapeutic care, of a keratin material, notably the skin, in particular facial skin, comprising at least the steps consisting in applying to said keratin material, notably the skin to be treated, a coat of at least one composition according to the invention.

According to the invention, the term “keratin materials” preferably refers to the skin, of the body, face and/or area around the eyes, the lips, the nails, mucous membranes, the eyelashes, the eyebrows, bodily hair, the scalp and/or head hair, or any other area of bodily skin.

More particularly, the keratin materials according to the invention are the scalp, the hair and/or the skin.

Preferably, the keratin material according to the invention is the skin.

The term “skin” refers to all of the skin of the body, and preferably the skin of the face, neckline, neck, arms and forearms, or even more preferably the skin of the face, notably of the forehead, nose, cheeks, chin and area around the eyes.

According to yet another of its aspects, the present invention relates to a process, notably a cosmetic process, for making facial skin matt and/or for reducing its shininess, comprising at least the step consisting in applying to the facial skin to be treated at least one composition according to the invention.

The term “making matt” means rendering the skin more matt, reducing the shininess thereof and therefore any unattractive highlights on the skin.

The invention also relates to the cosmetic use of the composition according to the invention for caring for combination and/or greasy skin.

### Hydrophobic Silica Aerogels

Silica aerogels are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York, Academic Press, 1990.

The hydrophobic silica aerogel particles used in the present invention preferably have a specific surface area per unit mass (S/M) ranging from 500 to 1500 m²/g, preferably from 600 to 1200 m²/g and better still from 600 to 800 m²/g, and a size expressed as the volume-mean diameter (D[0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

The specific surface area per unit mass may be determined by the nitrogen absorption method, known as the BET (Brunauer-Emmett-Teller) method, described in The Journal of the American Chemical Society, vol. 60, page 309, February 1938 and corresponding to international standard ISO 5794/1 (annex D). The BET specific surface area corresponds to the total specific surface area of the particles under consideration.

The sizes of the silica aerogel particles may be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is notably described in the publication by Van de Hulst, H. C., Light Scattering by Small Particles, Chapters 9 and 10, Wiley, New York, 1957.

According to an advantageous embodiment, the hydrophobic silica aerogel particles used in the present invention have a specific surface area per unit mass (SM) ranging from 600 to 800 m²/g.

The silica aerogel particles used in the present invention may advantageously have a tapped density ρ ranging from 0.02 g/cm³ to 0.10 g/cm³, preferably from 0.03 g/cm³ to 0.08 g/cm³ and in particular ranging from 0.05 g/cm³ to 0.08 g/cm³.

In the context of the present invention, this density, known as the tapped density, may be assessed according to the following protocol:

40 g of powder are poured into a graduated measuring cylinder and then the measuring cylinder is placed on a Stay 2003 device from Stampf Volumeter; the measuring cylinder is subsequently subjected to a series of 2500 packing actions (this operation is repeated until the difference in volume between two consecutive tests is less than 2%) and then the final volume Vf of packed powder is measured directly on the measuring cylinder.

The tapped density is determined by the ratio m/Vf, in this case 40/Vf (Vf being expressed in cm³ and m in g).

According to a preferred embodiment, the hydrophobic silica aerogel particles used in the present invention have a specific surface area per unit of volume S_{V} ranging from 5 to 60 m²/cm³, preferably from 10 to 50 m²/cm³ and better still from 15 to 40 m²/cm³.

The specific surface area per unit volume is given by the relationship: S_{V}=S_{M}×ρ; where ρ is the tapped density, expressed in g/cm³, and S_{M} is the specific surface area per unit mass, expressed in m²/g, as defined above.

Preferably, the hydrophobic silica aerogel particles used according to the invention have an oil absorption capacity, measured at the wet point, ranging from 5 to 18 ml/g, preferably from 6 to 15 ml/g and better still from 8 to 12 ml/g.

The absorption capacity, measured at the wet point and denoted Wp, corresponds to the amount of oil which it is necessary to add to 100 g of particles in order to obtain a homogeneous paste.

It is measured according to the “wet point” method or the method for determining the oil uptake of a powder described in standard NF T 30-022. It corresponds to the amount of oil adsorbed onto the available surface of the powder and/or absorbed by the powder by measurement of the wet point, described below:

An amount m=2 g of powder is placed on a glass plate and then the oil (isononyl isononanoate) is added dropwise. After addition of 4 to 5 drops of oil to the powder, mixing is performed using a spatula, and addition of oil is continued until conglomerates of oil and powder have formed. From this point, the oil is added at the rate of one drop at a time and the mixture is subsequently triturated with the spatula. The addition of oil is stopped when a firm and smooth paste is obtained. This paste must be able to be spread over the glass plate without cracks or the formation of lumps. The volume Vs (expressed in ml) of oil used is then noted.

The oil uptake corresponds to the ratio Vs/m.

The aerogels used according to the present invention are aerogels of hydrophobic silica, preferably of silylated silica (INCI name: silica silylate).

The term “hydrophobic silica” means any silica whose surface is treated with silylating agents, for example with halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

As regards the preparation of hydrophobic silica aerogel particles surface-modified by silylation, reference may be made to U.S. Pat. No. 7,470,725.

Use will preferably be made of hydrophobic silica aerogel particles surface-modified with trimethylsilyl groups, preferably having the INCI name Silica silylate.

As hydrophobic silica aerogels that may be used in the invention, an example that may be mentioned is the aerogel sold under the name VM-2260 or VM-2270 (INCI name: Silica silylate) by the company Dow Corning, the particles of which have a mean size of about 1000 microns and a specific surface area per unit mass ranging from 600 to 800 m²/g.

Mention may also be made of the aerogels sold by the company Cabot under the references Aerogel TLD 201, Aerogel OGD 201 and Aerogel TLD 203, Enova® Aerogel MT 1100 and Enova Aerogel MT 1200.

Use will preferably be made of the aerogel sold under the name VM-2270 (INCI name: Silica silylate) by the company Dow Corning, the particles of which have an average size ranging from 5-15 microns and a specific surface area per unit mass ranging from 600 to 800 m²/g.

The silica aerogel particles, in particular of silylated silica aerogel, may be present in a composition according to the invention in a content ranging from 0.5% to 13% by weight, preferably in a content ranging from 1% to 5% by weight and in particular ranging from 1% to 3% by weight relative to the total weight of the composition.

As stated previously, this aerogel material is combined with at least one ester hydrocarbon-based oil and at least one ether hydrocarbon-based oil.

For the purposes of the present invention, the term “oil” means any fatty substance that is in liquid form at room temperature (from 20 to 25° C.) at atmospheric pressure.

For the purposes of the invention, the term “hydrocarbon-based oil” covers a single hydrocarbon-based oil or a mixture of several hydrocarbon-based oils.

### Ester Hydrocarbon-based Oil

An ester hydrocarbon-based oil, also known as an ester oil, is an oil incorporating at least one ester function in its hydrocarbon-based structure. The ester hydrocarbon-based oils that are suitable for use in the invention may be of synthetic or plant origin. They are preferably non-volatile.

For the purposes of the present invention, the term “non-volatile oil” means an oil with a vapour pressure of less than 0.13 Pa at room temperature (from 20 to 25° C.) and atmospheric pressure.

The ester oils may be chosen from:

fatty acid esters, in particular of 4 to 22 carbon atoms, and notably of octanoic acid, heptanoic acid, lanolic acid, oleic acid, lauric acid or stearic acid, for instance propylene glycol dioctanoate, propylene glycol monoisostearate or neopentyl glycol diheptanoate;

synthetic esters, for instance the oils of formula R₁COOR₂ in which R₁ represents a linear or branched fatty acid residue including from 4 to 40 carbon atoms and R₂ represents a hydrocarbon-based chain, which is notably branched, containing from 4 to 40 carbon atoms, on condition that R₁+R₂≥16, for instance purcellin oil (cetostearyl octanoate), isononyl isononanoate, C₁₂ to C₁₅ alkyl benzoate, 2-ethylhexyl palmitate, octyldodecyl neopentanoate, 2-octyldodecyl stearate, 2-octyldodecyl erucate, oleyl erucate, isostearyl isostearate, 2-octyldodecyl benzoate, fatty alcohol or polyalcohol heptanoates, octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl myristate or 2-diethylhexyl succinate; preferably, the preferred synthetic esters R₁iCOOR₂ in which R₁ represents a linear or branched fatty acid residue including from 4 to 40 carbon atoms and R₂ represents a hydrocarbon-based chain, which is notably branched, containing from 4 to 40 carbon atoms are such that R₁ and R₂≥20;

linear fatty acid esters with a total carbon number ranging from 35 to 70, for instance pentaerythrityl tetrapelargonate (MW=697 g/mol);

glyceryl esters, for instance the caprylic/capric glyceride sold under the reference Capmul MCM by the company Abitec,

hydroxylated esters, preferably with a total carbon number ranging from 35 to 70, for instance polyglyceryl-2 triisostearate (MW=965 g/mol), isostearyl lactate, octyl hydroxystearate, octyldodecyl hydroxystearate, diisostearyl malate, glyceryl stearate; diethylene glycol diisononanoate;

esters of aromatic acids and of alcohols comprising 4 to 22 atoms, such as tridecyl trimellitate (MW=757 g/mol);

esters of C₂₄-C₂₈ branched fatty acids or fatty alcohols such as those described in patent application EP-A-0 955 039, and notably triisoarachidyl citrate (MW=1033.76 g/mol), pentaerythrityl tetraisononanoate (MW=697 g/mol), glyceryl triisostearate (MW=891 g/mol), glyceryl tris(2-decyl)tetradecanoate (MW=1143 g/mol), pentaerythrityl tetraisostearate (MW=1202 g/mol), polyglyceryl-2 tetraisostearate (MW=1232 g/mol) or pentaerythrityl tetrakis(2-decyl)tetradecanoate (MW=1538 g/mol);

polyesters resulting from the esterification of at least one triglyceride of hydroxylated carboxylic acid(s) with an aliphatic monocarboxylic acid and with an optionally unsaturated aliphatic dicarboxylic acid, for instance the succinic acid and isostearic acid castor oil sold under the reference Zenigloss by Zenitech, heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil or avocado oil, or else caprylic/capric acid triglycerides, for instance those sold by the company Stéarinerie Dubois or those sold under the names Miglyol 810, 812 and 818 by the company Dynamit Nobel, jojoba oil and shea butter oil;

esters of a diol dimer and of a diacid dimer of general formula HO—R¹—(—OCO—R²—COO—R¹—)ₕ—OH, in which:
R¹ represents a diol dimer residue obtained by hydrogenation of dilinoleic diacid,
R² represents a hydrogenated dilinoleic diacid residue, and
h represents an integer ranging from 1 to 9,
notably the esters of dilinoleic diacids and of dilinoleyl diol dimers sold by the company Nippon Fine Chemical under the trade names Lusplan DD-DA5® and DD-DA7®, and

polyesters obtained by condensation of an unsaturated fatty acid dimer and/or trimer and of diol, such as those described in patent application FR 0 853 634, in particular such as dilinoleic acid and 1,4-butanediol. Mention may notably be made in this respect of the polymer sold by Biosynthis under the name Viscoplast 14436H (INCI name: dilinoleic acid/butanediol copolymer), or else copolymers of polyols and of dimer diacids, and esters thereof, such as Hailucent ISDA.

The ester oils that are most particularly suitable for use in the invention are preferably chosen from:

liquid fatty acid triglycerides including from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, notably such as those sold by the company Stéarinerie Dubois or those sold under the names Miglyol 810, 812 and 818 by the company Dynamit Nobel, jojoba oil and shea butter oil;

synthetic esters, notably of fatty acids, for instance the oils of formulae R₁COOR₂ in which R₁ represents a fatty acid residue including from 8 to 29 carbon atoms and R₂ represents a branched or unbranched hydrocarbon-based chain containing from 3 to 30 carbon atoms, for instance purcellin oil, isononyl isononanoate, isopropyl myristate, isopropyl palmitate, 2-ethylhexyl palmitate, 2-octyldodecyl stearate, 2-octyldodecyl erucate or isostearyl isostearate;

hydroxylated esters, for instance isostearyl lactate, octyl hydroxystearate, octyldodecyl hydroxystearate, diisostearyl malate or triisocetyl citrate;

fatty alcohol heptanoates, octanoates or decanoates;

polyol esters, for instance propylene glycol dioctanoate, neopentyl glycol diheptanoate and diethylene glycol diisononanoate; and

pentaerythritol esters, for instance pentaerythrityl tetraisostearate.

Caprylic/capric acid triglycerides, isopropyl myristate, isopropyl palmitate, isononyl isononanoate and mixtures thereof are most particularly suitable for use in the invention.

In general, a composition according to the invention may contain from 1% to 15% by weight and preferably from 3% to 8% by weight of ester hydrocarbon-based oil relative to its total weight.

### Ether Hydrocarbon-based Oil

The ether hydrocarbon-based oil, also known as an ether oil, may be volatile or non-volatile and is preferably non-volatile.

An ether hydrocarbon-based oil is an oil of formula R₁OR₂ in which R₁ and R₂ independently denote a linear, branched or cyclic C₄-C₂₄ alkyl group, preferably a C₆-C₁₈ alkyl group, and preferably a C₈-C₁₂ alkyl group. It may be preferable for R₁ and R₂ to be identical.

Linear alkyl groups that may be mentioned include a butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group.

Branched alkyl groups that may be mentioned include a 1-methylpropyl group, a 2-methylpropyl group, a t-butyl group, a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group.

Cyclic alkyl groups that may be mentioned include a cyclohexyl group, a 3-methylcyclohexyl group and a 3,3,5-trimethylcyclohexyl group.

Advantageously, the ether oil is chosen from dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof.

Preferably, it is chosen from dicaprylyl ether, dicapryl ether, dilauryl ether, diisostearyl ether, dioctyl ether, and mixtures thereof.

Dicaprylyl ether is most particularly suitable for use.

In general, a composition according to the invention may contain from 2% to 20% by weight and preferably from 5% to 13% by weight of ether hydrocarbon-based oil relative to its total weight.

The mixture of the two oils required according to the invention is particularly efficient for significantly improving the sensory properties of the compositions according to the invention and in particular for ensuring that these properties are manifested when the compositions contain an amount of aerogel of greater than 1% by weight relative to their total weight, this amount being known to impair the soft and fluid sensation of compositions on application.

The combination of the three ingredients required according to the invention thus makes it possible to obtain an advantageous compromise between the desired matt effect and the expected sensory properties.

To do this, it is advantageous for a composition according to the invention to contain the ether and ester hydrocarbon-based oil components in an ether oil/ester oil weight ratio ranging from 1 to 2 and preferably from 1.2 to 1.8.

Advantageously, a composition according to the invention contains particles of hydrophobic silica aerogel, in particular of silylated silica aerogel, and dicaprylyl ether.

In particular, it contains more than 1% by weight or even 2% by weight of silica aerogel particles relative to its total weight.

In particular, it contains from 5% to 12% by weight of dicaprylyl ether relative to its total weight.

Preferably, this composition also contains a caprylic/capric acid triglyceride as ester oil, preferably in a content of from 4% to 8% by weight relative to its total weight.

### Other Components of a Composition According to the Invention

As stated previously, a composition according to the invention may be cosmetic and/or dermatological, preferably cosmetic.

The composition according to the invention is generally suitable for topical application to the skin and thus generally comprises a physiologically acceptable medium, i.e. a medium that is compatible with the skin. It is preferably a cosmetically acceptable medium, i.e. a medium which has a pleasant colour, odour and feel and which does not cause any unacceptable discomfort (stinging, tautness or redness) liable to discourage the user from applying this composition.

The composition according to the invention is in the form of an emulsion obtained by dispersing an oily phase in an aqueous phase (O/W).

The O/W emulsions according to the invention generally contain at least one emulsifier and optionally a co-emulsifier.

As emulsifiers that are suitable for use in the invention, examples that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof, such as the mixture of glyceryl stearate and PEG-40 stearate.

The emulsifier and the co-emulsifier are generally present in the composition in a proportion ranging from 0.3% to 20% by weight and preferably from 0.5% to 10% by weight relative to the total weight of the composition.

### Aqueous Phase

The aqueous phase comprises water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used according to the invention may also be volatile. Among the water-soluble solvents, mention may be made notably of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

Preferably, a composition according to the invention comprises at least one C₂-C₃₂ polyol.

For the purposes of the present invention, the term “polyol” should be understood as meaning any organic molecule including at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for use are those notably containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols, such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols, and mixtures thereof, and is preferably at least glycerol.

The aqueous phase is advantageously textured with a texturizer such as a thickener or gelling agent. Needless to say, the choice of this compound is made so as not to impair the other properties desired for the composition.

Gelling agents of carbomer type, for instance crosslinked acrylic acid homopolymers and copolymers, in particular those sold under the names Carbopol® 980, 981, 954, 2984 and 5984 by the company Goodrich or the products sold under the names Synthalen® M and Synthalen K by the company 3VSA, are notably suitable for use as texturizers.

A composition according to the invention may thus comprise from 0.3% to 1.5% by weight of gelling agent of crosslinked acrylic acid homopolymer and copolymer type relative to the total weight of the composition.

According to a preferred variant, a composition according to the invention contains at least one crosslinked acrylic acid copolymer.

According to another preferred variant, a composition according to the invention contains at least one sodium polyacrylate. It may notably be the product sold under the name Cosmedia SP from BASF.

Acrylamide homopolymers and copolymers acrylamidomethylpropanesulfonic acid copolymers also known as AMPS® are also suitable for use as gelling agents.

The aqueous phase may also be texturized with a compound of gum type, for instance xanthan gum.

A composition according to the invention may thus comprise from 0.05% to 2% by weight of gelling agent of gum type relative to the total weight of the composition.

Preferably, the aqueous phase represents from 40% to 80% by weight and preferably from 50% to 70% by weight relative to the total weight of said composition.

### Oily Phase

Besides the two oils and the hydrophobic aerogel material required according to the invention, the oily phase may comprise other compounds, provided, of course, that they do not harm the properties desired for the composition.

It may notably contain other additional oils or fatty substances.

On the other hand, and as mentioned previously, the oily phase is preferably free of silicone compounds and thus of silicone oil and/or elastomer.

These complementary oils may be volatile or non-volatile.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Among the volatile hydrocarbon-based oils containing from 8 to 16 carbon atoms, mention may be made notably of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. Preferably, the volatile hydrocarbon-based oil is chosen from volatile hydrocarbon-based oils containing from 8 to 16 carbon atoms, and mixtures thereof, in particular from isododecane, isodecane and isohexadecane, and is notably isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in Examples 1 and 2 of patent application WO 2008/155 059 from the company Cognis, and mixtures thereof.

As non-volatile hydrocarbon-based oil, mention may be made notably of fatty alcohols that are liquid at room temperature, with a branched and/or unsaturated carbon-based chain containing from 12 to 26 carbon atoms, for instance 2-octyldodecanol, isostearyl alcohol, oleyl alcohol and C₁₂-C₂₂ higher fatty acids, such as oleic acid, linoleic acid, linolenic acid, and mixtures thereof.

Preferably, the oily phase may be present in a content ranging from 3% to 55% by weight and preferably from 8% to 31% by weight relative to the total weight of said composition.

A cosmetic composition according to the invention may be in any presentation form conventionally used for topical application. These compositions are prepared according to the usual methods.

The compositions according to the invention may be more or less fluid and may have the appearance of a white or coloured cream, an ointment, a milk, a gel or a lotion.

Advantageously, the composition according to the invention has a pH ranging from 3 to 8. Preferably, the pH of the composition ranges from 4 to 6.

### Dyestuffs

The composition according to the invention may comprise at least one dyestuff (also known as a colouring agent), which may be chosen from water-soluble or liposoluble dyes, pigments and nacres, and mixtures thereof.

The presence of a dyestuff is more particularly advantageous for affording, in combination, a colouring makeup effect, but also coverage of the skin.

Thus, according to one variant, a composition according to the invention also contains at least one dyestuff, notably at least one pigment. According to this variant, a composition according to the invention may be in the form of a foundation.

In particular, a composition according to the invention may comprise one or more dyestuffs chosen from water-soluble dyes and pulverulent dyestuffs, for instance pigments, nacres and glitter flakes that are well known to those skilled in the art.

The dyestuffs may be present in the composition in a content ranging from 0.01% to 20% by weight, relative to the weight of the composition, preferably from 0.1% to 15% by weight.

The term “pigments” should be understood as meaning white or coloured, mineral or organic particles that are insoluble in an aqueous solution, which are intended to colour and/or opacify the resulting film.

The pigments may be present in a proportion of from 0.01% to 20% by weight, notably from 0.1% to 15% by weight and in particular from 0.2% to 10% by weight, relative to the total weight of the cosmetic composition.

As mineral pigments that may be used in the invention, mention may be made of titanium oxides, zirconium oxides or cerium oxides, and also zinc oxides, iron oxides or chromium oxides, ferric blue, manganese violet, ultramarine blue and chromium hydrate.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPPs) described in EP-A-542 669, EP-A-787 730, EP-A-787 731 and WO-A-96/08537.

The terms “nacres” should be understood as meaning coloured particles of any form, which may or may not be iridescent, notably produced by certain molluscs in their shell, or alternatively synthesized, and which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyestuffs. Examples of nacres that may also be mentioned include natural mica coated with titanium oxide, with iron oxide, with natural pigment or with bismuth oxychloride.

Among the nacres available on the market, mention may be made of the nacres Timica, Flamenco and Duochrome (based on mica) sold by the company Engelhard, the Timiron nacres sold by the company Merck, the Prestige mica-based nacres sold by the company Eckart, and the Sunshine synthetic mica-based nacres sold by the company Sun Chemical.

Preferably, the pigments and/or nacres may be present in the composition in a total content ranging from 0.01% to 20% by weight, relative to the weight of the composition, preferably from 0.1% to 15% by weight.

The term “dyes” should be understood as meaning compounds that are generally organic, which are soluble in fatty substances such as oils or in an aqueous-alcoholic phase.

The cosmetic composition according to the invention may also comprise water-soluble or liposoluble dyes. The liposoluble dyes are, for example, Sudan red, DC Red 17, DC Green 6, β-carotene, Sudan brown, DC Yellow 11, DC Violet 2, DC Orange 5 and quinoline yellow. The water-soluble dyes are, for example, beetroot juice or methylene blue.

Needless to say, according to another variant, a composition according to the invention is free of dyestuff and in particular of colour other than white.

According to this variant, a composition may notably be in the form of a white cream. The main purpose of such a composition is usually to afford a moisturizing effect in combination with the effect desired according to the invention.

A composition according to the invention may also contain various adjuvants commonly used in the cosmetics sector, such as fillers; preserving agents; sequestrants; fragrances; cosmetic active agents, for instance moisturizers, UV-screening agents, cicatrizing agents, agents for combating greasy skin, antipollution agents and/or skin anti-ageing agents.

Needless to say, a person skilled in the art will take care to select this or these optional additional compound(s), and/or the amount thereof, such that the advantageous properties of the composition according to the invention are not, or are not substantially, adversely affected by the envisioned addition.

Thus, in order to reinforce the matt effects of the composition according to the invention, it may notably comprise at least one active agent for caring for greasy skin.

In the context of the present invention, the term “active agent for caring for greasy skin” means a compound which, by itself, i.e. not requiring the intervention of an external agent to activate it, has biological activity. This active agent is preferentially chosen from desquamating agents, antimicrobial agents, anti-inflammatory agents, sebum regulators and antioxidants.

This or these active agent(s) may represent from 0.01% to 50%, preferably from 0.1% to 25% and better still from 0.5% to 10% by weight of the total weight of the composition.

Similarly, a composition according to the invention may comprise at least one moisturizer which may notably be chosen from sorbitol, polyhydric alcohols, preferably of C₂-C₈ and more preferably of C₃-C₆, preferably such as glycerol, propylene glycol, 1,3-butylene glycol, dipropylene glycol, diglycerol, glycerol, and mixtures thereof. According to a preferred embodiment, the moisturizer is glycerol.

The moisturizer is preferably present in the fatty phase in a content of between 0.1% and 10% by weight relative to the total weight of the composition.

The invention also relates to a cosmetic process for caring for keratin materials, in particular the skin, and more particularly for caring for facial skin, comprising the topical application to said keratin material and in particular to the skin of a composition according to the invention.

More specifically, said process is a process for making the skin matt and/or reducing its shininess.

The invention also relates to the cosmetic use of the composition according to the invention for caring for combination and/or greasy skin.

The invention will now be illustrated by the non-limiting examples that follow. In these examples, the amounts are indicated as weight percentages. The compounds are, depending on the case, cited as the chemical names or as the CTFA (International Cosmetic Ingredient Dictionary and Handbook) names.

### EXAMPLE 1

The compositions that follow were prepared according to the general procedure detailed below.

The starting materials (S/M) of A1 are melted at 70-75° C. A2 is then added just before emulsifying.

The SMs of phase D are mixed at room temperature by spatula.

The SMs of B are diluted while hot. Phase A is added thereto and the whole is homogenized for 5 minutes on a rotor/stator 3. The whole is diluted with the cold water of phase C with stirring using a rotor/stator 3.

Phase D is added and the whole is homogenized on a rotor/stator 3, neutralized with phase E and homogenized again on a deflocculator with cooling. The SMs of F are added thereto on a deflocculator with cooling, followed by phase G on a deflocculator.

Table 1 below gives the detailed composition of the two compositions thus obtained.

TABLE 1

| Phase | SUPPLIER | COMMERCIAL REF | EU INCI Name | No.1 | No.2 |
|---|---|---|---|---|---|
| A1 | GUJART ALKALIES | CAUSTIC LYE | POLYGLYCERYL-3 | 1.5 | 1.5 |
| | AND CHEMICAL S | | METHYLGLUCOSE | | |
| | | | DISTEARATE | | |
| A1 | EVONIK | TEGIN PELLETS | GLYCERYL | 1 | 1 |
| | GOLDSCHMIDT | | STEARATE SE | | |
| A1 | KAO | KALCOL 8098 | STEARYL ALCOHOL | 1 | 1 |
| A1 | DR STRAETMANS | DERMOSOFT OCTIOL | CAPRYLYL GLYCOL | 0.3 | 0.3 |
| A1 | ALTA LABORATORIES | SALICYLIC ACID USP | SALICYLIC ACID | 0.5 | 0.5 |
| A1 | CHIMEX | MEROXYL SAB | CAPRYLOYL | 0.3 | 0.3 |
| | | | SALICYLIC ACID | | |
| A1 | ECOGREEN | ROFETAN OE/MB | DICAPRYLYL ETHER | 7.5 | 11 |
| | OLEOCHEMICALS | | | | |
| A2 | ZMC (ZHEJIANG | 1300IU D-ALPHA- | TOCOPHEROL | 0.28 | 0.28 |
| | MEDICINE CO) | TOCOPHEROL | | | |
| | | (SOYBEAN OIL) | | | |
| A2 | IFF | 7222709 FIRST | FRAGRANCE | 0.3 | 0.3 |
| | | LOVE JK | | | |
| A3 | SACHTLEBEN | HOMBITAN FF | CI 77891 | | 4.1008 |
| | (HUNTSMAN) | PHARMA | | | |
| A3 | SUN | SUNPURO RED IRON | CI 77491 | | 0.2376 |
| | | OXIDE C33-8001 | | | |
| A3 | SUN | SUNPURO YELLOW | CI 77492 | | 0.528 |
| | | IRON OXIDE | | | |
| | | C33-9001 | | | |
| A3 | SUN | SUNPURO BLACKIRON | CI 77499 | | 0.132 |
| | | OXIDE C33-7001 | | | |
| A3 | ECKART | SYNAFIL S 1050 | SYNTHETIC | | 0.6816 |
| | | | FLUOROPHLOGOPITE | | |
| B | | | AQUA | 20 | 20 |
| B | ECOGREEN | ECOCEROL | GLYCERIN | 8 | 8 |
| | OLEOCHEMICALS | | | | |
| B | INNOSPEC ACTIVE | NATRLQUEST E30 | TRISODIUM | 0.25 | 0.25 |
| | CHEMICALS | | ETHYLENEDIAMINE | | |
| | | | DISUCCINATE | | |
| C | | | AQUA | QS. | QS. |
| | | | | 100 | 100 |
| D | CREMER OLEO (IOI) | MIGLYOL 812 N | CAPRYLIC/CAPRIC | 5 | 6.5 |
| | | | TRIGLYCERIDE | | |
| D | 3V | SYNTHALEN K | CARBOMER | 0.9 | |
| | BASF | COSMEDIA SP | SODIUM POLYCARYLATE | | 1 |
| D | RHODIA (SOLVAY) | RHODICARE XC | XANTHAN GUM | 0.1 | 0.1 |
| E | OLIN | CAUSTIC SODA 50% | SODIUM HYDROXIDE | 0.3 | 0.11 |
| F | DOW CORNING | DOW CORNING | | | |
| | (DOW CHEMICAL) | VM-2270 | SILICA SILYLATE | 2 | 2 |
| | | AEROGEL FINE | | | |
| | | PARTICLES | | | |
| F | ROQUETTE | BEAUTE-BY- | ZEA MAY STARCH | 1 | 0 |
| | | ROQUETTE ST005 | | | |
| G | FRANCE ALCOOLS | ETHANOL S96 DENATURE | ALCOHOL DENAT. | 5 | 5 |
| | | BITREX/TERTIO | | | |
| | | | TOTAL | 100 | 100 |

### EXAMPLE 2

A composition according to the invention is prepared according to the protocol detailed in Example 1. This composition was tested for its matt-effect power and evaluated for its sensory qualities.

Control compositions were also prepared to undergo the same tests. In terms of galenical formulation, they reproduce that of composition 1, but in which the 7.5% of dicaprylyl ether and the 5% of caprylic/capric triglyceride were replaced with another oil. Table 2 below states, for each control composition, which other compound replaced these two components of composition 1 and in what amount.

### Test to Evaluate the Matt-effect Power

The gloss of a deposit resulting from the application of a composition may be commonly measured according to various methods, such as the method using a Byk Micro TRI gloss 60° glossmeter.

Principle of the Measurement using this Glossmeter

The machine illuminates the sample to be analysed at a certain incidence and measures the intensity of the specular reflection.

The intensity of the reflected light depends on the material and on the angle of illumination. For non-ferrous materials (paint, plastic), the intensity of reflected light increases with the angle of illumination. The rest of the incident light penetrates the material and, depending on the shade of the colour, is either partly absorbed or scattered.

The reflectometer measurement results are not based on the amount of incident light but on a polished black glass standard of defined refractive index.

The measurement is normalized relative to an internal standard and brought to a value out of 100. For this calibration standard, the measurement value is set at 100 gloss units (calibration).

The closer the measured value is to 100, the more glossy the sample. The measurement unit is the Gloss Unit (GU).

The angle of illumination used has a strong influence on the reflectometer value. In order to be able to readily differentiate very glossy and matt surfaces, the standardization has defined 3 geometries or 3 measurement domains.

### Test Protocol

a—On a contrast card (Prufkarte type 24/5-250 cm²) sold by the company Erichsen, the composition whose mean gloss it is desired to evaluate is spread at a rate of 2 mg/cm² using a mechanical film spreader to obtain a film 100 microns thick. The coat covers the white background and the black background of the card.

b—Leave to dry for 24 hours at 37° C.

c—Measure the gloss at 60° on the absorbent white background

(3 measurements) using a Byk Gardner brand glossmeter of reference microTri-Gloss.

1 spray of a mixture (20% oleic acid+80% Vichy water+1% Oleth-10) is applied; the amount deposited is about 0.3 g per spray.

The reflection is measured using a gonioreflectometer (immediate gloss in GU), after 12 minutes at room temperature (gloss persistence in GU). The result obtained is the ratio R between the specular reflection and the scattered reflection (gloss unit or GU). The value of R is proportionally smaller the greater the matt effect.

The gloss assessment scale is as follows:

−: glossy

0: no effect

+: sparingly matt

++: matt

+++: very matt

TABLE 2

| | | | | Mattness test |
|---|---|---|---|---|
| Reference 1 formula | | | | T12 min |
| % MP | EU INCI | SUPPLIER | COMMERCIAL REF. | Gloss unit |
| 75% | DICAPRYLYL ETHER | BASF | CETIOL OE | ++ |
| 5% | CAPRYLIC/CAPRIC | CREMER OLEO | MIGLYOL 812 N | |
| (No. 1 according | TRIGLYCERIDE | (IOI) | | |
| to the invention) | | | | |
| 12.5% | CAPRYLIC/CAPRIC | CREMER OLEO | MIGLYOL 812 N | + |
| (comparative) | TRIGLYCERIDE | (IOI) | | |
| 12.5% | ISOPROPYL PALMITATE | BASF | ISOPROPYL | + |
| (comparative) | | | PALMITATE | |
| 12.5% | ISONONYL | ALZO | WICKENOL 151 | + |
| (comparative) | ISONONANOATE | | | |
| 12.5% | COCO-CAPRYLATE/ | BASF | CETIOL C 5C | + |
| (comparative ) | CAPRATE | | | |
| 12.5% | SQUALANE | CLARIANT | PLANTASENS OLIVE | − |
| (comparative) | | | SQUALANE | |
| 12.5% | ISOCETYL STERATE | STEARINERIE | ISOCETYL | − |
| (comparative) | | DUBOIS | STEARATE (DUB | |
| | | | SIS16) | |
| 12.5% | HYDROGENATED | NOF | PARLEAM | − |
| (comparative) | POLYISOBUTENE | CORPORATION | | |
| 12.5% | DICAPRYLYL | BASF | CETIOL CC | − |
| (comparative) | CARBONATE | | | |
| 12.5% | OLEA EUROPAEA FRUIT | OLVEA | VIRGIN OLIVE OIL | − |
| (comparative) | OIL | | | |
| 12.5% | BRASSICA CAMPESTRIS | HENRY | RAPESEED OIL, | − |
| (comparative) | SEED OIL | LAMOTTE | REFINED | |
| 12.5% | HELIANTHUS ANNUUS | HUILERIES DE | REFINED | − |
| (comparative) | SEED OIL | LAPALISSE | SUNFLOWER OIL | |

It may be noted that only the composition according to the invention makes it possible to obtain the desired efficacy.

### Tests to Evaluate the Sensory Qualities

The glidance and tack qualities are assessed, respectively, during and after topical application of a sample of the test composition to the surface of the back of a hand.

The glidance is assessed by evaluating the ease with which the fingers slide on the surface of the back of a hand during the application to this surface of a sample of the test composition. To reinforce the tactile evaluation, the application is performed using two fingers which are made to slide on the surface.

Glidance Key:

0: dragging

+: glidant

++: very glidant

The tackiness is evaluated by touch, by tapping the skin with the application fingers.

Tackiness Key:

−: not tacky

0: no effect

+: sparingly tacky

++: tacky

+++: very tacky

TABLE 3

| | | | | SENSORY | |
|---|---|---|---|---|---|
| % MP | EU INCI | SUPPLIER | COMMERCIAL REF. | glidant | tacky |
| 75% | DICAPRYLYL ETHER | BASF | CETIOL OE | ++ | 0 |
| 5% | CAPRYLIC/CAPRIC | CREMER OLEO | MIGLYOL 812 N | | |
| (No. 1 according | TRIGLYCERIDE | (IOI) | | | |
| to the invention) | | | | | |
| 12.5% | CAPRYLIC/CAPRIC | CREMER OLEO | MIGLYOL 812 N | + | 0 |
| (comparative) | TRIGLYCERIDE | (IOI) | | | |
| 12.5% | ISOPROPYL PALMITATE | BASF | ISOPROPYL | + | 0 |
| (comparative) | | | PALMITATE | | |
| 12.5% | ISONONYL | ALZO | WICKENOL 151 | + | 0 |
| (comparative) | ISONONANOATE | | | | |
| 12.5% | COCO-CAPRYLATE/ | BASF | CETIOL C 5C | ++ | ++ |
| (comparative ) | CAPRATE | | | | |
| 12.5% | SQUALANE | CLARIANT | PLANTASENS OLIVE | + | ++ |
| (comparative) | | | SQUALANE | | |
| 12.5% | ISOCETYL STERATE | STEARINERIE | ISOCETYL | + | ++ |
| (comparative) | | DUBOIS | STEARATE (DUB | | |
| | | | SIS16) | | |
| 12.5% | HYDROGENATED | NOF | PARLEAM | ++ | ++ |
| (comparative) | POLYISOBUTENE | CORPORATION | | | |
| 12.5% | DICAPRYLYL | BASF | CETIOL CC | + | 0 |
| (comparative) | CARBONATE | | | | |
| 12.5% | OLEA EUROPAEA FRUIT | OLVEA | VIRGIN OLIVE OIL | 0 | ++ |
| (comparative) | OIL | | | | |
| 12.5% | BRASSICA CAMPESTRIS | HENRY | RAPESEED OIL, | + | ++ |
| (comparative) | SEED OIL | LAMOTTE | REFINED | | |
| 12.5% | HELIANTHUS ANNUUS | HUILERIES DE | REFINED | + | ++ |
| (comparative) | SEED OIL | LAPALISSE | SUNFLOWER OIL | | |

From the results, it emerges that only the composition comprising the combination of an ester hydrocarbon-based oil and an ether hydrocarbon-based oil according to the invention displays the desired effects.

1. Composition, which is in the form of an oil-in-water, O/W, emulsion, and comprising at least
a) particles of at least one hydrophobic silica aerogel,
b) an ester hydrocarbon-based oil, and
c) an ether hydrocarbon-based oil,
in which said ether and ester oils are present in an ether oil/ester oil weight ratio ranging from 1 to 2.

2. The composition according to claim 1, in which the hydrophobic silica aerogel particles have a specific surface area per unit mass ranging from 500 to 1500 m²/g, and a size expressed as the volume mean diameter (D[0.5]) ranging from 1 to 1500 μm.

3. The composition according to claim 1, in which the hydrophobic silica aerogel particles have an oil absorption capacity, measured at the wet point, ranging from 5 to 18 ml/g of particles.

4. The composition according to claim 1, in which the hydrophobic silica aerogel particles are particles of at least one silylated silica aerogel (INCI name: silica silylate).

5. The composition according to claim 1, wherein the hydrophobic silica aerogel particles represent, as solids, from 0.5% to 13% by weight relative to the total weight of the composition.

6. The composition according to claim 1, wherein it contains from 1% to 15% by weight of ester hydrocarbon-based oil relative to the total weight of the composition.

7. The composition according to claim 1, wherein said ester hydrocarbon-based oil is selected from the group consisting of:
liquid fatty acid triglycerides including from 4 to 10 carbon atoms;
synthetic esters;
hydroxylated esters;
fatty alcohol heptanoates, octanoates or decanoates;
polyol esters; and
pentaerythritol esters.

8. The composition according to claim 1, wherein said ester hydrocarbon-based oil is selected from the group consisting of caprylic/capric acid triglycerides, isopropyl myristate, isopropyl palmitate, isononyl isononanoate, and mixtures thereof.

9. The composition according to claim 1, comprising from 2% to 20% by weight of ether hydrocarbon-based oil relative to the total weight of the composition.

10. The composition according to claim 1, wherein said ether hydrocarbon-based oil is selected from the group consisting of dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof.

11. The composition according to claim 1, further comprising at least one polyol.

12. The composition according to claim 1, further comprising from 0.3% to 1.5% by weight of gelling agent of crosslinked acrylic acid homopolymer and copolymer type relative to the total weight of the composition.

13. The composition according to claim 1, wherein the aqueous phase represents from 40% to 80% by weight relative to the total weight of said composition.

14. The composition according to claim 1, comprising less than 1% by weight, of silicone oil and/or of silicone elastomer.

15. The composition according to claim 1, further comprising a dyestuff.

16. The composition according to claim 1, which is free of dyestuff.

17. Cosmetic process for caring for a keratin material, comprising the topical application to said keratin material to be treated of a composition according to claim 1.

18. Process for making facial skin matt and/or for reducing shininess of facial skin, comprising the topical application to the skin to be treated of a composition according to claim 1.

19. A method for caring for combination and/or greasy skin comprising applying a composition according to claim 1.

20. The composition according to claim 11, wherein the polyol is selected from the group consisting of ethylene glycol, pentaerythritol, trimethylolpropane, propylene glycol, 1,3-propanediol, butylene glycol, isoprene glycol, pentylene glycol, hexylene glycol, glycerol, polyglycerols, polyethylene glycols, and mixtures thereof.

21. The composition according to claim 1. which is free of silicone oil and/or of silicone elastomer.
